(19) United States
(12) Patent Application Publication
(10) Pub. No.: US 2015/0121441 A1
(43) Pub. Date: Apr. 30, 2015

(54) SYSTEMS AND METHODS FOR EMBEDDING MULTIMEDIA DATA IN SURGICAL FEEDS

(71) Applicants: Prashant Apte, Mumbai (IN); Suresh Deshpande, Kolhapur (IN)

(72) Inventors: Prashant Apte, Mumbai (IN); Suresh Deshpande, Kolhapur (IN)

(21) Appl. No.: 14/589,147

(22) Filed: Jan. 5, 2015

Related U.S. Application Data
(63) Continuation of application No. PCT/IN2013/000408, filed on Jul. 3, 2013.

(30) Foreign Application Priority Data
Jul. 5, 2012 (IN) 1943/MUM/2012

(51) Int. Cl. G06F 19/00; H04N 21/4223; H04N 5/77; H04N 21/4788; H04N 21/854; H04N 21/231; H04N 21/472

(52) U.S. Cl. CPC G06F 19/3481; G06F 19/3418; H04N 21/231; H04N 21/4223; H04N 21/47205; H04N 21/4788; H04N 21/854; H04N 5/77; A61B 2019/5214
USPC 725/109

(57) ABSTRACT

Systems and methods for embedding and sharing video feeds captured during a surgery, to users, in real-time, including video receivers for receiving video feed from video recording equipment, a video stream distribution server receiving the video feed, a relay transferring the video feed to users by rendering the video feed in relation to bandwidth available to each of the users and selecting a protocol of RTP, RTSP, and RTMP and selecting transmission parameters of video bit rate parameters, frames per second parameters, window size(s) parameters, buffer size parameters, key frame interval parameters. Systems and methods create and transmit video feeds with full duplex communication, viewing options, and surgery-performable video frame sequence transmission at substantially reduced bandwidth between origin and end user.

### RELATED APPLICATIONS

This application claims priority under 35 U.S.C. §120 to, and is a continuation of, co-pending International Application PCT/IN2013/000408, filed Jul. 3, 2013 and designating the US, which claims priority to Indian Application 1943/MUM/2012, filed Jul. 5, 2012, such Indian Application also being claimed priority to under 35 U.S.C. §119. These Indian and International applications are incorporated by reference herein in their entireties.

### FIELD

This invention relates to the field of video streaming, information systems, relational databases, and conferencing platforms.

Example embodiments include systems, methods, and platforms, for embedding video feeds captured during a surgery, potentially in real-time.

### BACKGROUND

Capturing moments, actions, events, tasks, emotions, and the like evolved from drawings to still pictures by cameras to a series of still pictures conjuring up a motion picture using cameras suited for such purposes. Cameras evolved from taking still photographs on film to moving images on film to still photographs in digital format to moving images on digital format. The captured videos or series of images or pictures or photographs were earlier stored on film (in negative format) and developed in order to make copies (in positive format). Storage of such captured videos or series of images or pictures or photographs changed to digital format on hard disks, pen drives, and memory cards. In order to facilitate sharing in online mode, sharing sites cropped up which allow user to upload captured videos or series of images or pictures or photographs and then allow them to share it with other users through Internet.

Due to increase in bandwidth by Internet Service Providers, videos could be captured and transmitted in real-time from one geographical location on this planet to another geographical location on this planet. This provisioned video conferencing. Videoconferencing is the conduct of a videoconference (also known as a video-conference or video-teleconference) by a set of telecommunication technologies which allow two or more locations to communicate by simultaneous two-way video and audio transmissions.

Further, there have been advances in video capturing, video editing, video streaming, video transmitting systems and methods. One of the areas where videos or such systems and methods are currently used is in surgeries. Typically, in a laparoscopic surgery, a scope (camera) which records visual information is introduced in a human body, through one portal, which visual information is transmitted to a local display unit so that the operating surgeon can see the internal are of the operating body on the display unit. Other portals are used to introduce surgical instruments to perform surgery whilst the surgeon views the surgery through the display unit.

The output of the camera can be used in multiple manners. However, there is a need for a system, method, and platform, which allows real-time sharing of video feed with multiple people, remotely located. Further, there is a need for a system, method, and platform, which allows the multiple people to access the video feed, to comment on the video feed, to correspond with the video feed, to interact with the video feed, to embed the video feed with video input, audio input, text input, hyperlinked input, multimedia input, communication input, market input, and the like. Also, there is a need for a system, method, and platform, which allows a user to access the video feed as it is or in its embedded or changed format as and when required. Further, there is a need for a system, method, and platform to impart privacy control settings to the various embedding features of a captured video stream.

### SUMMARY

Example embodiments include a system for embedding and sharing video feeds captured during a surgery, to participating users, in real-time, said system comprises:

a video receiving means adapted to receive at least a video feed from at least a video recording equipment handled by a video feed originator;

a video stream distribution server adapted to receive said video feed from said video receiving means;

a relaying mechanism adapted to relay said video feed to said participating users, said relaying mechanism comprises a rendering mechanism adapted to render said video feed in relation to bandwidth available at each of said participating users, in that, said rendering mechanism comprises a protocol selection means adapted to select a protocol from a group of protocols consisting of RTP, RTSP, RTMP protocols and said rendering mechanism further comprises a transmission parameters' selection means adapted to select transmission parameters from a group of transmission parameters consisting of video bit rate parameters, frames per second parameters, window size(s) parameters, buffer size parameters, key frame interval parameters, thereby enabling full duplex communication, viewing options, surgery-performable video frame sequence transmission at substantially reduced bandwidth between said originator of said video feed and said at least a participating user viewing said video feed.

### BRIEF DESCRIPTION OF THE DRAWINGS

Example embodiments will become more apparent by describing, in detail, the attached drawings, wherein like elements are represented by like reference numerals, which are given by way of illustration only and thus do not limit the terms which they depict.

FIG. 1 illustrates a schematic block diagram of the system, method, and platform;

FIG. 2 illustrates an indexed video sequence in terms of steps; and

FIG. 3 illustrates backtransmitted video feed between a video feed originator and user in picture-in-picture format.

### DETAILED DESCRIPTION

Because this is a patent document, general broad rules of construction should be applied when reading it. Everything described and shown in this document is an example of subject matter falling within the scope of the claims, appended below. Any specific structural and functional details disclosed herein are merely for purposes of describing how to make and use example embodiments. Several different embodiments not specifically disclosed herein may fall within the claim scope; as such, the claims may be embodied in many alternate forms and should not be construed as limited to only example embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, “and” and “or” are equivalent to the term “and/or,” which includes any and all combinations of one or more of the associated listed items.

It will be understood that when an element is referred to as being “connected,” “coupled,” “mated,” “attached,” or “fixed” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). Similarly, a term such as “communicatively connected” includes all variations of information exchange routes between two devices, including intermediary devices, networks, etc., connected wirelessly or not.

As used herein, the singular forms “a”, “an” and “the” and the plural form “indicia” are intended to include both the singular and plural forms, unless the language explicitly indicates otherwise. It will be further understood that the terms comprises,” “comprising,” “includes,” and/or “including,” when used herein, specify the presence of stated features, integers, steps, operations, elements, and/or components, but do not themselves preclude the presence or addition of one or more other features, integers, steps, operations, elements, components, and/or groups thereof.

It should also be noted that the structures and operations discussed below may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, so as to provide looping or other series of operations aside from the single operations described below. It should be presumed that any embodiment having features and functionality described below, in any workable combination, falls within the scope of example embodiments.

An object of some example embodiments is to provide a system, method, and platform for capturing video feed from a laparoscopic camera during surgery, and to store the captured video feed on an online portal in an authorised accessible format.

Another object of some example embodiments is to provide a system, method, and platform for capturing video feed from a laparoscopic camera during surgery, and to store the captured video feed on an online portal in an authorised accessible format, in a real-time manner.

Yet another object of some example embodiments is to provide a system, method, and platform for capturing video feed from a laparoscopic camera during surgery, and to allows real-time sharing of video feed with multiple people, remotely located.

Still another object of some example embodiments is to provide a system, method, and platform, for capturing video feed from a laparoscopic camera during surgery, and to allow multiple people to access the video feed, to comment on the video feed, to correspond with the video feed, to interact with the video feed.

An additional object of some example embodiments is to provide a system, method, and platform, for capturing video feed from a laparoscopic camera during surgery, and to allow multiple people to embed the video feed with video input, audio input, text input, hyperlinked input, multimedia input, communication input, market input, and the like.

Yet an additional object of some example embodiments is to provide a system, method, and platform, for capturing video feed from a laparoscopic camera during surgery, and to allow multiple people to embed the video feed with video input, audio input, text input, hyperlinked input, multimedia input, communication input, market input, and the like, which system, method, and platform allows a user to access the video feed as it is or in its embedded or changed format as and when required.

Still an additional object of some example embodiments is to provide a system, method, and platform, for capturing video feed from a laparoscopic camera during surgery, and to allow multiple people to embed the video feed with video input, audio input, text input, hyperlinked input, multimedia input, communication input, market input, and the like, which system, method, and platform is adapted to impart privacy control settings to the various embedded features of a captured video stream.

Another additional object of some example embodiments is to provide a system, method, and platform, for capturing video feed from a laparoscopic camera during surgery, and to allow relatively newer/lesser experienced surgeons to use it for training and information purposes.

Yet another additional object of some example embodiments is to provide a system, method, and platform, for capturing video feed from a laparoscopic camera during surgery, and to allow a surgeon to extend their expertise to a wider audience.

Still another additional object of some example embodiments is to provide a system, method, and platform, for capturing video feed from a laparoscopic camera during surgery, and to allow collaboration of other participating users during a surgery.

Another object of some example embodiments is to provide a system, method, and platform, for capturing video feed from a laparoscopic camera during surgery, and to allow assistance of other participating users during a surgery in audio, video, text, marker, multimedia, communication, and the like format.

Yet another object of some example embodiments is to provide a system, method, and platform, for capturing video feed from a laparoscopic camera during surgery, and to provide training material (surgery videos) embellished with audio, video, marker, text, communication, and the like captions.

Example embodiments include a system, method, and platform, for embedding and sharing video feeds. Particularly, a system, method, and platform, for embedding and sharing video feeds, in real-time captured during a surgery. Example embodiments may include relays, servers, video equipment, multiplexors, and display devices including computer processors and attendant memory and busses that are specifically configured to execute example methods and described functionality, through both software and hardware configurations.

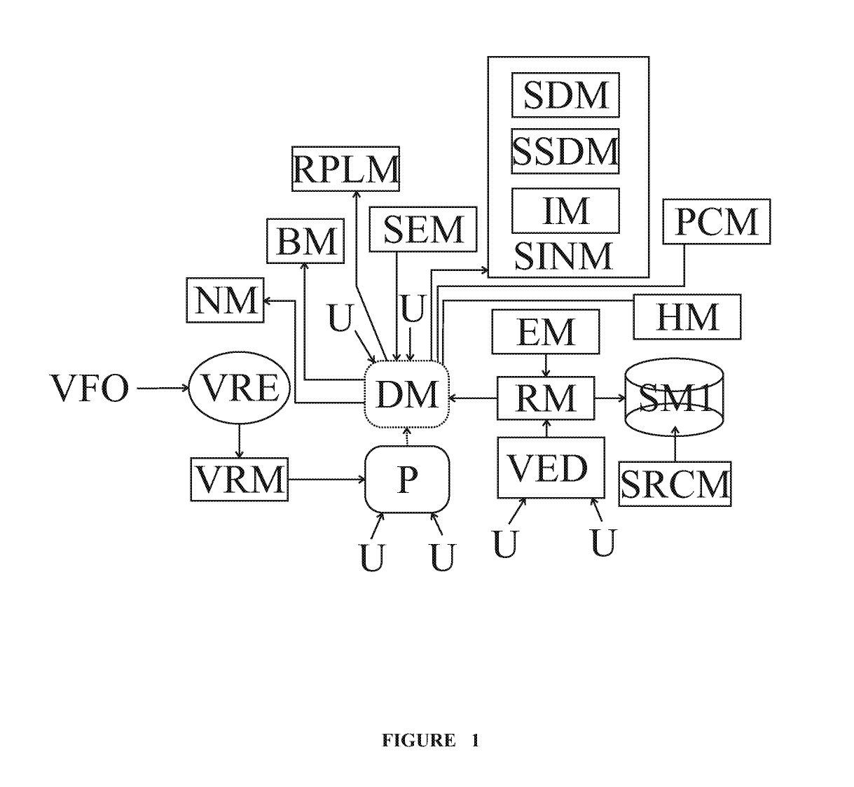

FIG. 1 illustrates a schematic block diagram of the system, method, and platform.

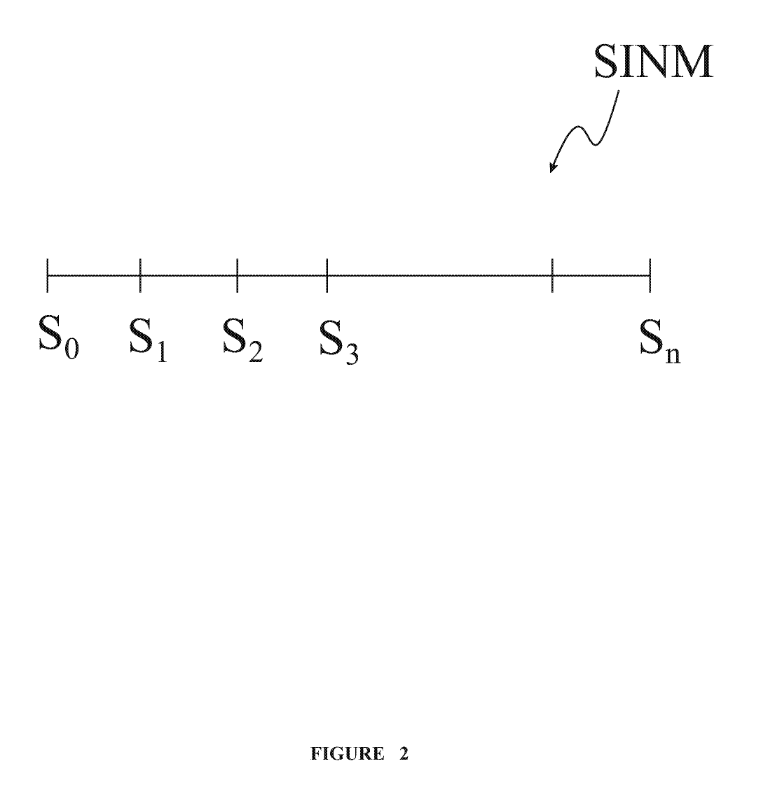

FIG. 2 illustrates an indexed video sequence in terms of steps.

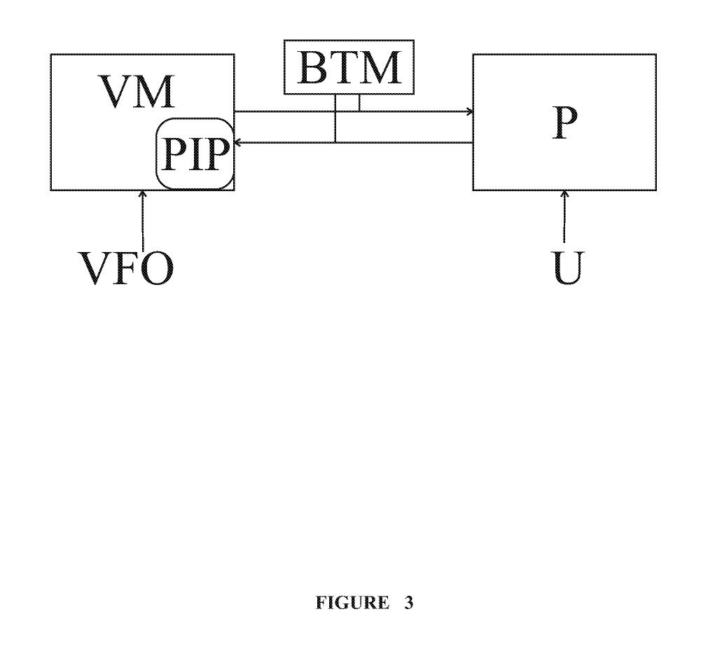

FIG. 3 illustrates backtransmitted video feed between a video feed originator and user in picture-in-picture format.

In accordance with an example embodiment, there is provided a video receiving means (VRM) adapted to receive at least a video feed from video recording equipment (VRE). This video receiving means may be a real-time video receiving means, buffered video receiving means, time delayed video receiving means, offset video receiving means, storage-enabled video receiving means, cache-enabled video receiving means, or the like. The video recording equipment is adapted to be handled by a video feed originator (VFO) such as surgeon or assistant.

According to a non-limiting exemplary embodiment, the video recording means may be a camera used in surgery, such as a laparoscopic camera. The video feed originator, typically, may be a surgeon performing a surgery using a video recording equipment such as a scope.

The system and method uses RTP, RTSP, RTMP protocols. Typical variables are video bitrate, frames per second (fps), window size, buffer size, key frame interval. Depending on the camera being used to transmit and the expected output the values above need to be modified. Typical ranges for the above variables are as follows:

Video bit rate: 300 kbps to 10 Mbps

Frames per second: from 15 to 60

Window size(s): 320×240, 1024×768, 1920×1280

Buffer size: from 1 second to 3 seconds

Key frame interval: from 3 to 25.

The video receiving means acts as a transmitter of video stream or as a viewer. It connects to a media server using any of the protocols, above. The data in the stream is stored on the server and distributed to all connections waiting to view the stream.

In accordance with another example embodiment, there is provided a video stream distribution server (P) to receive video feed from the video receiving means. Alternatively, the video receiving means is the video stream distribution server.

The video receiving means, typically, is communicably coupled to the video stream distribution server/portal on the Internet.

Participating users (U) can access the portal. Also, the person at the location from where video is being captured shall use the portal to view the video feed. The participating user may be a surgeon who is relaying the video feed, too.

According to a non-limiting exemplary embodiment, preferably, this video receiving means is communicably coupled to a camera used in surgery. This surgery may be laparoscopic surgery. The output of camera is transmitted to the video receiving means and on to a portal connected to the Internet. Typically, this is real-time transmission and real-time uploading. Thus, a live surgery can be transmitted. A person, having access to the portal, can watch the surgery.

Participating users can view the video in real-time, through the portal. Even, the surgeon performing the surgery views the video through the portal.

In accordance with yet another example embodiment, there is provided a video embedding dashboard (VED) adapted to allow a user to embed a video on the portal or that is being relayed through the portal. This embedding is done in real-time. Embedding relates to embellishing the video feed with additional content. Embedding relates to adding additional content to the video feed. A plurality of tools, means, and mechanisms are provided in order to facilitate such embedding. Preferably, the embedding is done in real-time such that the surgeon viewing the video feed is able to view the embedded video. Additionally, the embedding may be done in offline mode. The embedding dashboard allows a user to tag a video feed at discrete intervals of time. The tagging may include audio tagging, video tagging, text tagging, or multimedia tagging. Further, the tagging may include drawing tagging allowing a user to draw on images of the video feed in real time. The video embedding dashboard is, typically, enabled with a relaying mechanism (RM) adapted to relay said video feed to said participating users, said relaying mechanism comprising a rendering mechanism adapted to render said video feed in relation to bandwidth available at each of said participating users, in that, said rendering mechanism comprising a protocol selection means adapted to select a protocol from a group of protocols consisting of RTP, RTSP, RTMP protocols and said rendering mechanism further comprising a transmission parameters' selection means adapted to select transmission parameters from a group of transmission parameters consisting of video bit rate parameters, frames per second parameters, window size(s) parameters, buffer size parameters, key frame interval parameters. This enables full duplex communication, viewing options, surgery-performable video frame sequence transmission at substantially reduced bandwidth between said originator of said video feed and said at least a participating user viewing said video feed. The relaying mechanism is further adapted to relay the tagged objects and embedded video in real-time to participating users. Also, the video embedding dashboard is, typically, enabled with a storage mechanism (SM1) adapted to store the video and tagged objects and embedded video. The video embedding dashboard (VED) is, further, typically, enabled with an editing mechanism (EM) adapted to edit the video feed. The video embedding dashboard (VED) is adapted to allow a user to embed stored pictures or pointers as overlay frames on said video feed. The overlay frames may be in a transparent or translucent fashion and provides as reference images on a live organ video feed in order to help define the organ visible in the video feed. The overlay frames may therefore serve as reference images.

The video stream distribution server (P) may include a duplication means (DM) adapted to duplicate the video feed in real time. The video embedding dashboard may allow embedding and/or editing on the duplicated video feed. This allows a user where video feed is originated to choose viewing of a clean video feed and/or embedded/edited video feed, alternatively or simultaneously.

The video stream distribution server (P) comprises a cache memory/database in order to store received video feed.

Hence, the video can be embedded with:

audio inputs: additional audio inputs can be tagged in a time referenced manner or in an image referenced manner or without any reference;

video inputs: additional video inputs can be tagged in a time referenced manner or in an image referenced manner or without any reference;

text inputs: additional text inputs can be tagged in a time referenced manner or in an image referenced manner or without any reference;

multimedia inputs: additional multimedia inputs can be tagged in a time referenced manner or in an image referenced manner or without any reference;

marker inputs: additional marker or freehand inputs can be tagged in a time referenced manner or in an image referenced manner or without any reference.

Thus, the system, method, and platform provides full duplex communication between the originator of video feed and the participants or users viewing the video feed.

Also, the system, method, and platform provided an embedded video, which embedding may be text and/or audio/video/multimedia/hyperlink embedding such that theoretical information about a surgery and relevant videos, tagged thereof, will be available to a user/surgeon at the time of scheduling a surgery in order to look out for standard steps in a procedure, pitfalls, and things to lookout for, relevant diagrams, relevant videos, notes, and the like.

Further, the system and method are adapted to incorporate multiple bit-rate transmission. This allows for access to the system and method even if the users' accessing portal has various connection speeds.

The system and method are aimed towards surgeons or participating entities, specifically towards laparoscopic/endoscopic surgeons from different geographies. Amongst other constraints, the constraints that may impact transmitting or viewing of surgeries are the resolution of the recording equipment being used and the bandwidth available (for transmission and viewing). It is common knowledge that typically the trainers will have access to state of the art equipment (good HD camera, high bandwidth lease line etc) whereas the consumers (viewers) especially in rural areas may not have that kind of luxury. Hence, it is of utmost importance to ensure feasibility of infrastructure between the transmitter and the viewer. E.g., the transmitter may decide to use a HD camera at its full potential and transmit the output at 4 Mbps but the viewer may have a shared 1 Mbps connection which obviously will not be sufficient to have a good viewing experience. The solution to achieve compatibility, hence, is to encode the input stream at multiple bit rates and feed every viewer with the best fit based on pre-defined parameters. E.g., a 4 Mbps stream should also be encoded at various other bit rates right from 300 Kbps to 4 Mbps. When a user connects, the system shall identify the bandwidth available with the user and intelligently decide to push the stream with the appropriate bit rate. This will ensure that all viewers, participating entities, devoid of the bandwidth that they have will get a smooth viewing experience.

According to a non-limiting exemplary embodiment, a surgery video feed being captured from a camera during surgery can be embedded with:

audio inputs: additional audio inputs can be tagged in a time referenced manner or in an image referenced manner or without any reference. A surgeon transmitting the video can also transmit audio. Similarly, users watching the video can communicate with the surgeon through audio. Each of these audio inputs is stored at a particular video position.

video inputs: additional video inputs can be tagged in a time referenced manner or in an image referenced manner or without any reference.

text inputs: additional text inputs can be tagged in a time referenced manner or in an image referenced manner or without any reference. Users watching the video can add text inputs. Each of these are stored in a database with a specific reference to the video position.

multimedia inputs: additional multimedia inputs can be tagged in a time referenced manner or in an image referenced manner or without any reference.

marker inputs: additional marker or freehand inputs can be tagged in a time referenced manner or in an image referenced manner or without any reference. Users can draw (free hand drawing) on the video to identify specific things, similar to markers.

All the embedded videos are stored on a storage database. Videos transmitted using the process above can be accessed along with the audio and text inputs as well as the markers that were drawn on the video. Clicking on the audio/text inputs will start the video from the position when the audio/text inputs were inserted during the real time streaming session.

Thus, newer or upcoming surgeons who do not have sufficient opportunities to watch live surgeries can use these videos for training. This can ensure the training of surgeons even when they are not present in the operation theatre. Also, surgeons in small towns/villages who do not have exposure of handling complex surgeries can get help from expert surgeons in a real-time and aided and guided manner.

In accordance with still another example embodiment, there is provided a surgery index navigator means (SINM) in order to allow a user/surgeon to navigate through a video feed in accordance with multiple choices of navigation. The surgery index navigator comprises a surgery defining means (SDM) adapted to define a surgery or type of surgery. Names of surgeries may be fed into the surgery defining means. The surgery index navigator further comprises a surgery step defining means (SSDM) adapted to defined steps of defined surgeries. Each surgery or type of surgery includes a number of steps. These steps are quantified and qualified and jotted down in to the step defining means. The steps may be time-indexed, time-referenced, image-indexed, image-referenced. Each step may be linked/linked/embedded to/with additional text data, video data, audio data, multimedia data, hyperlinked data; which can be recalled or fetched upon prompt of user/viewer. The surgery index navigator further comprises an indexing means (IM) adapted to allow a user/surgeon to index a video sequence in terms of steps. This can be seen in FIG. 2 of the accompanying drawings. Reference numerals S1, S2, S3, . . . Sn refer to indexed steps in a video frame. Thus, we get an indexed step-wise timeline in a video sequence. The video sequence is partitioned, wherein, each partition is defined by a start point and an end point thereby providing a defined step, thereof. A user/surgeon can review the surgical procedure, step-wise, by correlating the steps in the video sequence with the steps defined by the step defining means. This review aids in determining if all steps have been conducted, sequentially. Typically, its use is of paramount importance in a surgery of a longer duration. The surgery index navigator further comprises a replay mechanism (RPLM) in order to provide live replays during the video streaming, itself. The provisioning of time-indexed or time-referenced or image-indexed or image-referenced steps allows for a search mechanism to search for specific steps of a video feed (or a surgery).

In accordance with an additional example embodiment, there is provided a secondary embedding means (SEM) adapted to allow a viewer to embed in a video feed. This embedding may be audio embedding, video embedding, text embedding, or multimedia embedding. There is a first transmission of the video feed from a user (or surgeon) to a viewer. The secondary embedding means is equipped with a back transmission means (BTM) adapted to allow back transmission of data to the video feed originator's (or surgeon) viewing mechanism (VM) or participating user's (U) viewing mechanism. This back transmitted data can be viewed in an overlapped format, picture-in-picture format, split view format, or the like format at the user's (or surgeon's) viewing mechanism. The picture-in-picture (PIP) backtransmitted video feed is seen in FIG. 3 of the accompanying drawings.

In accordance with yet an additional example embodiment, there is provided a broadcasting mechanism (BM) adapted to broadcast defined points of a video feed or defined intervals of a video feed on to various other sharing or networking portals on the Internet. A snapshot or a series of snapshots or videos can thus be selectively broadcasted. This enables updating in real time. The broadcasting mechanism comprises social media broadcasts such as twitter broadcasts, facebook broadcasts, google plus broadcasts and the like in a real-time manner.

In accordance with still an additional example embodiment, there is provided a searching means (SRCM) adapted to search a video in accordance with pre-defined parameters of searching. These pre-defined parameters of searching may include a graphical search by anatomy, geography, location, defined steps, and the like.

In accordance with another additional example embodiment, there is provided a privacy control mechanism (PCM) adapted to provide transmission rights of video feed. According to an exemplary embodiment, in case a user/viewer wants to transmit during emergency surgery, a transmitter can give him/her one time access to transmit.

In accordance with yet another additional example embodiment, there is provided a help means (HM) adapted to store defined steps in an indexed manner. The defined steps could be linked/tagged/embedded with additional data. The help means lists steps and its links and can be used to fetch/prompt/recall details.

According to a non-limiting exemplary embodiment, the system, method, and platform may provide a gall bladder image with all the arteries and veins in and around it. When a surgeon is operating and cannot make out the different arteries (or other organs) around the gall bladder, he will fetch or pull up the reference image or video of the gall bladder section image and align it with the actual gall bladder of the patient (or its video feed). The image, because, it has all the other parts of the anatomy around the gall bladder will snap fit and the surgeon will now be able to identify the patients artery based on the position of the artery in the image. Each of these anatomy parts will be transparent and will be labeled for ease of use and understanding by the surgeon.

In accordance with another example embodiment, a notification means (NM) is provided adapted to allow a first user who provides the video feed to invite or notify other users to view the video feed on the portal provided by the system, method, and platform. The notification means may work with a scheduler for automatic scheduling and notification.

The system and the method provide the ability to tag a video with audio or text or video or multimedia or combinations, thereof; by both the transmitter and the viewer. Also, it is possible to select specific audio streams during playback (stored video played at a later point in time). E.g., if there are 10 participating entities (surgeons) who have given their views during a LIVE surgery, a first user or moderator or authorised personnel shall have the control to select only a few of those views and only those audio streams will get played back along with the stored video. Further, the system and method shall provide capabilities to draw on the video.

Typically, said parameters' selection means adapted to select transmission parameters from a group of transmission parameters consisting of video bit rate parameters is selected from a bit rate of 300 kbps to 10 Mbps.

Typically, said parameters' selection means adapted to select transmission parameters from a group of transmission parameters consisting of frames per second is selected from 15 frames per second to 60 frames per second.

Typically, said parameters' selection means adapted to select transmission parameters from a group of transmission parameters consisting of window size(s) is selected from window sizes of 320×240, 1024×768, or 1920×1280 pixels.

Typically, said parameters' selection means adapted to select transmission parameters from a group of transmission parameters consisting of buffer size is selected from 1 second to 3 seconds

Typically, said parameters' selection means adapted to select transmission parameters from a group of transmission parameters consisting of key frame interval is selected from 3 to 25.

Typically, said system comprises a video embedding dashboard adapted to allow a user to embed content in to/on to said received video on said video stream distribution server or that is being relayed through said video stream distribution server and further adapted to allow at least one of said participating user to tag said video feed at least a discrete interval of time.

Typically, said video receiving means is selected from a group of video receiving means consisting of real-time video receiving means, buffered video receiving means, time delayed video receiving means, offset video receiving means, storage-enabled video receiving means, cache-enabled video receiving means, and its combinations.

Typically, said video recording means is a camera selected from a group of cameras consisting of laparoscopic camera, endoscopic camera, and any external camera in an operation theater.

Typically, said video recording means is the video stream distribution server.

Alternatively, said video receiving means, typically, is communicably coupled to the video stream distribution server on the Internet.

Typically, said video stream distribution server is accessible to participating users including video feed originator.

Additionally, said system comprises a video embedding dashboard adapted to allow a user to embed said video feed on said video stream distribution server, said embedding being done in real-time or offline mode.

Additionally, said system comprises a video embedding dashboard adapted to allow a user to tag said video feed at discrete intervals of time, said tagging comprises audio tagging, video tagging, text tagging, multimedia tagging, drawing tagging allowing a user to draw on images of said video feed in real time.

Additionally, said system comprises a video embedding dashboard adapted to allow a user to embed stored pictures and pointers as overlay frames on said video feed.

Additionally, said relaying mechanism being further adapted to relay tagged objects and embedded video in real-time to participating users.

Additionally, said video embedding dashboard is enabled with a storage mechanism adapted to store video and tagged objects and embedded video.

Additionally, said video embedding dashboard is enabled with an editing mechanism adapted to edit said video feed.

Additionally, said video stream distribution server comprises duplication means adapted to duplicate said video feed in real time such that said video embedding dashboard being adapted to embed and/or edit on said duplicated video feed, thereby allowing a video feed originator to choose viewing of a clean video feed and/or embedded/edited video feed, alternatively or simultaneously.

Additionally, said video stream distribution server comprises a cache memory/database in order to store received said video feed.

Additionally, said system comprises a video embedding dashboard adapted to embed a video feed with audio inputs such that additional audio inputs can be tagged in a time referenced manner or in an image referenced manner or without any reference.

Additionally, said system comprises a video embedding dashboard adapted to embed a video feed with video inputs such that additional video inputs can be tagged in a time referenced manner or in an image referenced manner or without any reference.

Additionally, said system comprises a video embedding dashboard adapted to embed a video feed with text inputs such that additional text inputs can be tagged in a time referenced manner or in an image referenced manner or without any reference.

Additionally, said system comprises a video embedding dashboard adapted to embed a video feed with multimedia inputs such that additional multimedia inputs can be tagged in a time referenced manner or in an image referenced manner or without any reference.

Additionally, said system comprises a video embedding dashboard adapted to embed a video feed with marker inputs such that additional marker or freehand inputs can be tagged in a time referenced manner or in an image referenced manner or without any reference.

Typically, said system being a multiple bit-rate transmission system in order to allow for access to said system even if said participating users' accessing portal has various connection speeds.

Typically, said system comprises a surgery index navigator means in order to allow a user/surgeon to navigate through said video feed in accordance with multiple choices of navigation, said surgery index navigator further comprises:

surgery defining means adapted to define a surgery or type of surgery;

surgery step defining means adapted to define steps of said defined surgeries; and

indexing means adapted to allow a user/surgeon to index said video feed in terms of said defined steps in order to obtain an indexed step-wise timeline in said video feed.

Alternatively, said system comprises a surgery index navigator means in order to allow a user/surgeon to navigate through said video feed in accordance with multiple choices of navigation, said surgery index navigator further comprises:

surgery defining means adapted to define a surgery or type of surgery;

surgery step defining means adapted to define steps of said defined surgeries, characterised in that, said steps being time-indexed, time-referenced, image-indexed, or image-referenced; and

indexing means adapted to allow a user/surgeon to index said video feed in terms of said defined steps in order to obtain an indexed step-wise timeline in said video feed.

Alternatively, said system comprises a surgery index navigator means in order to allow a user/surgeon to navigate through said video feed in accordance with multiple choices of navigation, said surgery index navigator further comprises:

surgery defining means adapted to define a surgery or type of surgery;

surgery step defining means adapted to define steps of said defined surgeries, characterised in that, each of said steps being linked or embedded to/with additional text data, video data, audio data, multimedia data, hyperlinked data; which can be recalled or fetched upon prompt of user/viewer; and

indexing means adapted to allow a user/surgeon to index said video feed in terms of said defined steps in order to obtain an indexed step-wise timeline in said video feed.

Alternatively, said system comprises a surgery index navigator means in order to allow a user/surgeon to navigate through said video feed in accordance with multiple choices of navigation, said surgery index navigator further comprises:

surgery defining means adapted to define a surgery or type of surgery;

surgery step defining means adapted to define steps of said defined surgeries; and

indexing means adapted to allow a user/surgeon to index said video feed in terms of said defined steps in order to obtain an indexed step-wise timeline in said video feed, said video feed being partitioned, wherein, each partition is defined by a start point and an end point thereby providing a defined step, thereof in order to allow a user/surgeon to review a surgical procedure, step-wise, by correlating said steps in said video feed with the steps defined by said step defining means.

Alternatively said system comprises a surgery index navigator means in order to allow a user/surgeon to navigate through said video feed in accordance with multiple choices of navigation, said surgery index navigator further comprises:

surgery defining means adapted to define a surgery or type of surgery;

surgery step defining means adapted to define steps of said defined surgeries, characterised in that, said steps being time-indexed, time-referenced, image-indexed, or image-referenced;

indexing means adapted to allow a user/surgeon to index said video feed in terms of said defined steps in order to obtain an indexed step-wise timeline in said video feed; and

replay mechanism in order to provide live replays during the video streaming, itself.

Alternatively, said system comprises a surgery index navigator means in order to allow a user/surgeon to navigate through said video feed in accordance with multiple choices of navigation, said surgery index navigator further comprises:

surgery defining means adapted to define a surgery or type of surgery;

surgery step defining means adapted to define steps of said defined surgeries, characterised in that, said steps being time-indexed, time-referenced, image-indexed, or image-referenced; and

indexing means adapted to allow a user/surgeon to index said video feed in terms of said defined steps in order to obtain an indexed step-wise timeline in said video feed; and

search mechanism adapted to allow mechanism to search for specific steps of a video feed depending upon said time-indexed parameters or time referenced parameters or image-indexed parameters or image-referenced parameters.

Typically, said system comprises a secondary embedding means adapted to allow a viewer or said participating user to embed in a video feed, said embedding being audio embedding, video embedding, text embedding, or multimedia embedding.

Typically, said system comprises a secondary embedding means adapted to allow a viewer or said participating user to embed in a video feed, said embedding being audio embedding, video embedding, text embedding, or multimedia embedding, characterised in that, said secondary embedding means being equipped with a back transmission means adapted to allow back transmission of data to a video feed originator's portal.

Typically, said system comprises a broadcasting mechanism adapted to broadcast defined points of said video feed or defined intervals of said video feed on to various other sharing or networking portals on the Internet.

Typically, said system comprises a searching means adapted to search a video in accordance with pre-defined parameters of searching.

Typically, said system comprises a privacy control mechanism adapted to provide transmission rights of video feed.

Typically, said system comprises a help means adapted to store defined steps in an indexed manner, said defined steps being linked/tagged/embedded with additional data such that said help means provides listing of steps and its links and can be used to fetch/prompt/recall details.

Typically, said system comprises a notification means adapted to allow said video feed originator to invite or notify other users to view said video feed on a portal provided by said system.

Example methods include embedding and sharing video feeds captured during a surgery, to participating users, in real-time, said method comprises the steps of:

receiving at least a video feed, using a video receiving means, from at least a video recording equipment handled by a video feed originator;

receiving said video feed, using a video stream distribution server, from said video receiving means;

relaying said video feed to said participating users, using a relaying mechanism, said step of relaying comprises a step of rendering said video feed in relation to bandwidth available at each of said participating users, using a rendering mechanism, in that, said step of rendering comprises a step of selecting a protocol from a group of protocols, using protocol selection means, consisting of RTP, RTSP, RTMP protocols and said step of rendering further comprises a step of selecting transmission parameters, using transmission parameters' selection means, from a group of transmission parameters consisting of video bit rate parameters, frames per second parameters, window size(s) parameters, buffer size parameters, key frame interval parameters; thereby, enabling full duplex communication, viewing options, surgery-perfomable video frame sequence transmission at substantially reduced bandwidth between said originator of said video feed and said at least a participating user viewing said video feed.

Typically, said step of selecting transmission parameters comprises a step of selecting transmission parameters from a group of transmission parameters consisting of video bit rate parameters is selected from a bit rate of 300 kbps to 10 Mbps.

Typically, said step of selecting transmission parameters comprises a step of selecting transmission parameters from a group of transmission parameters consisting of frames per second is selected from 15 frames per second to 60 frames per second.

Typically, said step of selecting transmission parameters comprises a step of selecting transmission parameters from a group of transmission parameters consisting of window size(s) is selected from window sizes of 320×240, 1024×768, or 1920×1280 pixels.

Typically, said step of selecting transmission parameters comprises a step of selecting transmission parameters from a group of transmission parameters consisting of buffer size is selected from 1 second to 3 seconds

Typically, said step of selecting transmission parameters comprises a step of selecting transmission parameters from a group of transmission parameters consisting of key frame interval is selected from 3 to 25.

Typically, said method comprises a step of allowing a user to embed content in to/on to said received video on said video stream distribution server or that is being relayed through said video stream distribution server, using a video embedding dashboard, and further comprises a step of allowing at least one of said participating user to tag said video feed at least a discrete interval of time.

Typically, said step of receiving said video feed comprises a step of selecting said video receiving means from a group of video receiving means consisting of real-time video receiving means, buffered video receiving means, time delayed video receiving means, offset video receiving means, storage-enabled video receiving means, cache-enabled video receiving means, and its combinations.

Typically, said video recording means is selected from a group of cameras consisting of laparoscopic camera, endoscopic camera, and any external camera in an operation theater.

Typically, said video recording means is the video stream distribution server.

Alternatively, said video receiving means, typically, is communicably coupled to the video stream distribution server on the Internet.

Typically, said video stream distribution server is accessible to participating users including video feed originator.

Additionally, said method comprises a step of allowing a user to embed said video feed on said video stream distribution server, said embedding being done in real-time or offline mode, using video embedding dashboard.

Additionally, said method comprises a step of allow a user to tag said video feed at discrete intervals of time, said tagging comprises audio tagging, video tagging, text tagging, multimedia tagging, drawing tagging allowing a user to draw on images of said video feed in real time, using video embedding dashboard.

Additionally, said method comprises a step of allowing a user to embed stored pictures and pointers as overlay frames on said video feed, using video embedding dashboard.

Additionally, said step of relaying said video feed to said participating users comprises a step of further relay tagged objects and embedded video in real-time to participating users, using said relaying mechanism.

Additionally, said method comprises a step of allowing a user to embed content in to/on to said received video on said video stream distribution server or that is being relayed through said video stream distribution server, using a video embedding dashboard, said step of allowing a user to embed content comprises a step of storing video and tagged objects and embedded video, using a storage mechanism.

Additionally, said method comprises a step of allowing a user to embed content in to/on to said received video on said video stream distribution server or that is being relayed through said video stream distribution server, using a video embedding dashboard, said step of allowing a user to embed content comprises a step of editing said video feed, using an editing mechanism.

Additionally, said receiving said video feed, using a video stream distribution server, comprises a step of duplicating said video feed in real time, using duplication means, such that step of duplicating couples with a step of embedding and/or editing on said duplicated video feed, using video embedding dashboard, thereby allowing a video feed originator to choose viewing of a clean video feed and/or embedded/edited video feed, alternatively or simultaneously.

Additionally, said step of receiving said video feed, using a video stream distribution server, comprises a step of storing received said video feed, using a cache memory/database.

Additionally, said method comprises a step of embedding a video feed with audio inputs such that additional audio inputs can be tagged in a time referenced manner or in an image referenced manner or without any reference, using video embedding dashboard.

Additionally, said method comprises a step of embedding a video feed with video inputs such that additional video inputs can be tagged in a time referenced manner or in an image referenced manner or without any reference, using video embedding dashboard.

Additionally, said method comprises a step of embedding a video feed with text inputs such that additional text inputs can be tagged in a time referenced manner or in an image referenced manner or without any reference, using video embedding dashboard.

Additionally, said method comprises a step of embedding a video feed with multimedia inputs such that additional multimedia inputs can be tagged in a time referenced manner or in an image referenced manner or without any reference, using video embedding dashboard.

Additionally, said method comprises a step of embedding a video feed with marker inputs such that additional marker or freehand inputs can be tagged in a time referenced manner or in an image referenced manner or without any reference, using video embedding dashboard.

Typically, said method being a multiple bit-rate transmission method in order to allow for access to said method even if said participating users' accessing portal has various connection speeds.

Typically, said method comprises a step of allowing a user/surgeon to navigate through said video feed, using surgery index navigator means, in accordance with multiple choices of navigation, said step further comprises the steps of:

defining a surgery or type of surgery, using surgery defining means;

defining steps of said defined surgeries, using surgery step defining means; and

allowing a user/surgeon to index said video feed in terms of said defined steps in order to obtain an indexed step-wise timeline in said video feed, using indexing means.

Alternatively, said method comprises a step of allowing a user/surgeon to navigate through said video feed, using surgery index navigator means, in accordance with multiple choices of navigation, said step further comprises the steps of:

defining a surgery or type of surgery, using surgery defining means;

defining steps of said defined surgeries, using surgery step defining means, characterised in that, said steps being time-indexed, time-referenced, image-indexed, or image-referenced; and

allowing a user/surgeon to index said video feed in terms of said defined steps in order to obtain an indexed step-wise timeline in said video feed, using indexing means.

Alternatively, said method comprises a step of allowing a user/surgeon to navigate through said video feed, using surgery index navigator means, in accordance with multiple choices of navigation, said step further comprises the steps of:

defining a surgery or type of surgery, using surgery defining means;

defining steps of said defined surgeries, using surgery step defining means, characterised in that, each of said steps being linked or embedded to/with additional text data, video data, audio data, multimedia data, hyperlinked data; which can be recalled or fetched upon prompt of user/viewer; and

allowing a user/surgeon to index said video feed in terms of said defined steps in order to obtain an indexed step-wise timeline in said video feed, using indexing means.

Alternatively, said method comprises a step of allowing a user/surgeon to navigate through said video feed, using surgery index navigator means, in accordance with multiple choices of navigation, said step further comprises the steps of:

defining a surgery or type of surgery, using surgery defining means;

defining steps of said defined surgeries, using surgery step defining means; and

allowing a user/surgeon to index said video feed in terms of said defined steps in order to obtain an indexed step-wise timeline in said video feed, using indexing means, said video feed being partitioned, wherein, each partition is defined by a start point and an end point thereby providing a defined step, thereof in order to allow a user/surgeon to review a surgical procedure, step-wise, by correlating said steps in said video feed with the steps defined by said step defining means.

Alternatively, said method comprises a step of allowing a user/surgeon to navigate through said video feed, using surgery index navigator means, in accordance with multiple choices of navigation, said step further comprises the steps of:

defining a surgery or type of surgery, using surgery defining means;

defining steps of said defined surgeries, using surgery step defining means, characterised in that, said steps being time-indexed, time-referenced, image-indexed, or image-referenced;

allowing a user/surgeon to index said video feed in terms of said defined steps in order to obtain an indexed step-wise timeline in said video feed, using indexing means; and

providing live replays during the video streaming, itself, using replay mechanism.

Alternatively, said method comprises a step of allowing a user/surgeon to navigate through said video feed, using surgery index navigator means, in accordance with multiple choices of navigation, said step further comprises the steps of:

defining a surgery or type of surgery, using surgery defining means;

defining steps of said defined surgeries, using surgery step defining means, characterised in that, said steps being time-indexed, time-referenced, image-indexed, or image-referenced; and

allowing a user/surgeon to index said video feed in terms of said defined steps in order to obtain an indexed step-wise timeline in said video feed, using indexing means; and

searching for specific steps of a video feed, using search mechanism, depending upon said time-indexed parameters or time referenced parameters or image-indexed parameters or image-referenced parameters.

Typically, said method comprises a step of allowing a viewer or said participating user to embed in a video feed, using secondary embedding, said embedding being audio embedding, video embedding, text embedding, or multimedia embedding.

Typically, said method comprises a step of allowing a viewer or said participating user to embed in a video feed, using secondary embedding, said embedding being audio embedding, video embedding, text embedding, or multimedia embedding, characterised in that, said secondary embedding means being equipped with a back transmission means adapted to allow back transmission of data to a video feed originator's portal.

Typically, said method comprises a step of broadcasting defined points of said video feed or defined intervals of said video feed on to various other sharing or networking portals on the Internet, using broadcasting mechanism.

Typically, said method comprises a step of searching a video in accordance with pre-defined parameters of searching, using searching means.

Typically, said method comprises a step providing transmission rights of video feed using privacy control mechanism.

Typically, said system method a step of storing defined steps in an indexed manner, said defined steps being linked/tagged/embedded with additional data such that said help means provides listing of steps and its links and can be used to fetch/prompt/recall details, using help means.

Typically, said method comprises a step of allowing said video feed originator to invite or notify other users to view said video feed on a portal provided by said method, using notification means.

Some example embodiments being described here, it is understood that one or more examples and components thereof may be used in combination and/or in duplication to produce multiple options and functionalities for multi-media surgical feeds. It will further be appreciated by one skilled in the art that example embodiments may be varied through routine experimentation and without further inventive activity. For example, although a real-time information overlay is used in some examples, it is understood that video recording, consumption, and/or editing with relevant media may equally be executed with large passages of time therebetween in other examples. Variations are not to be regarded as departure from the spirit and scope of the exemplary embodiments, and all such modifications as would be obvious to one skilled in the art are intended to be included within the scope of the following claims.

What is claimed is:

1. A system for embedding and sharing video feeds captured during a surgery with users, the system comprising:
a video receiver configured to receive a video feed from a video recording equipment;
a video stream distribution server configured to receive the video feed from the video receiver; and
a relay configured to relay the video feed to the users, wherein the relay includes a renderer configured to render the video feed in relation to bandwidth available to each of the users, and wherein the renderer includes,
a protocol selector configured to select at least one of RTP, RTSP, and RTMP protocols, and
a transmission parameter selector configured to select at least one of video bit rate, frames per second, window size(s), buffer size, and key frame interval parameters, wherein the video feed is performed at a bandwidth reduced for each of the users and includes full duplex communication, viewing options, and a surgery-performable video frame sequence transmission.

2. The system of claim 1, wherein the transmission parameter selector is configured to select at least one of,
a video bit rate of about 300 kbps to 10 Mbps,
a frames per second from about 15 frames per second to 60 frames per second,
a window size from 320×240, 1024×768, and 1920×1280 pixels,
a buffer size from about 1 second to 3 seconds, and
a key frame interval from about 3 to 25.

3. The system of claim 2, wherein the transmission parameter selector is configured to select all of the video bit rate, the frames per second, the window size, the buffer size, and the key frame interval.

4. The system of claim 1, further comprising:
a video embedding dashboard configured to receive input from each of the users to embed the video feed on the video stream distribution server in real-time or offline mode, wherein the dashboard is configured to receive input from each of the users to tag the video feed at discrete intervals of time with audio tagging, video tagging, text tagging, multimedia tagging, and drawing tagging.

5. The system of claim 1, further comprising:
a video embedding dashboard configured to embed the video feed with audio, video, text, multimedia, marker, and freehand inputs in a time-referenced manner, an image-referenced manner, or without any reference.

6. The system of claim 5, wherein the video embedding dashboard includes a store for storing video.

7. The system of claim 1 wherein the receiver, relay, and server are configured for multiple bit-rate transmissions so as to permit access by the users having varying connection speeds.

8. The system of claim 1, wherein the video receiver is configured for at least one of real-time video receiving, buffered video receiving, time-delayed video receiving, offset video receiving, storage-enabled video receiving, and cache-enabled video receiving.

9. The system of claim 1, further comprising:
a surgery index navigator including surgery definitions, surgery step definitions, and an index configured to retrieve input from the users to index time-steps of the video feed based on the surgery definitions and surgery step definitions.

10. The system of claim 9, wherein the surgery step definitions are at least one of time-indexed, time-referenced, image-indexed, and image-referenced.

11. The system of claim 10, wherein the surgery step definitions are linked to or embedded with at least one of text, video, audio, multimedia, or hyperlinks accessible to the users.

12. A method of embedding and sharing video feeds captured during a surgery with users, the method comprising:
receiving, with a video receiver, video feed from a video recording equipment;
receiving, with a video stream distribution server, the video feed from the video receiver;
selecting, with a relay, at least one of RTP, RTSP, and RTMP protocols and at least one of video bit rate, frames per second, window size(s), buffer size, and key frame interval parameters; and
relaying, with the relay, the video feed in relation to bandwidth available to each of the users, at a bandwidth reduced for each of the users, and including full duplex communication, viewing options, and a surgery-performable video frame sequence transmission.

13. The method of claim 12, wherein the selecting includes selecting at least one of,
a video bit rate of about 300 kbps to 10 Mbps,
a frames per second from about 15 frames per second to 60 frames per second,
a window size from 320×240, 1024×768, and 1920×1280 pixels,
a buffer size from about 1 second to 3 seconds, and
a key frame interval from about 3 to 25.

14. The method of claim 13, wherein the selecting includes selecting all of the video bit rate, the frames per second, the window size, the buffer size, and the key frame interval.

15. The method of claim 12, further comprising:
embedding, with a video embedding dashboard, the video feed on the video stream distribution server in real-time or offline mode; and
tagging, with the video embedding dashboard, the video feed at discrete intervals of time with audio tagging, video tagging, text tagging, multimedia tagging, and drawing tagging.

16. The method of claim 12, further comprising:
embedding, with a video embedding dashboard, the video feed with audio, video, text, multimedia, marker, and freehand inputs in a time-referenced manner, an image-referenced manner, or without any reference.

17. The method of claim 12, wherein the first receiving, the second receiving, the selecting, and the relaying are executed for multiple bit-rate transmissions so as to permit access by the users having varying connection speeds.

18. The method of claim 12, wherein the video receiver is configured for at least one of real-time video receiving, buffered video receiving, time-delayed video receiving, offset video receiving, storage-enabled video receiving, and cache-enabled video receiving.

19. The method of claim 12, further comprising:
indexing time-steps of the video feed with surgery definitions, surgery step definitions.

20. The method of claim 19, wherein the surgery step definitions are at least one of time-indexed, time-referenced, image-indexed, and image-referenced, and wherein the surgery step definitions are linked to or embedded with at least one of text, video, audio, multimedia, or hyperlinks accessible to the users.
